(19) United States
(12) Patent Application Publication
(10) Pub. No.: US 2010/0221333 A1
(43) Pub. Date: Sep. 2, 2010

(54) Pellets Comprising a Core With a Water-Soluble Carrier

(76) Inventors: Burkard Schlutermann, Au (DE); Manfred Kohlmeyer, Basel (CH); Jan Schroer, Hauenstein (DE)

Correspondence Address: HEDMAN & COSTIGAN, P.C., 1230 AVENUE OF THE AMERICAS, 7th floor, NEW YORK, NY, 10020, US

(21) Appl. No.: 12/086,933

(22) PCT Filed: Dec. 21, 2005
(86) PCT No.: PCT/EP2005/013798

§ 371 Date: Apr. 7, 2010

(51) Int. Cl. A61K 9/14; A61K 9/20; A61P 43/00

(52) U.S. Cl. 424/465; 424/489

(57) ABSTRACT

A pellet with a length/width ratio of less than about 1.4 is provided which comprises a core which comprises a water-soluble carrier and an active ingredient distributed homogeneously therein. The pellet disintegrates, with visually ascertainable loss of the pellet structure, into smaller fragments when the following test is carried out: (a) 200 to 800 mg of pellets are placed into a Petri dish with 0.5 ml of demineralized water. (b) The Petri dish is immediately afterwards fixed to the measuring cylinder holder of a stamping volumeter. (c) After 10 seconds, 30 stamping movements are carried out. (d) Immediately afterwards, the pellets are photographed.

The invention relates to an oral pharmaceutical preparation and a method of production thereof. In particular the invention relates to a preparation that is based on spherical pellets, which contain a water-soluble excipient and an active substance. The invention also relates to neutral pellets, which can be used for the production of pharmaceutical preparations.

Production of pharmaceutical pellets by providing conventional starter cores with coatings that contain active substances is known. Furthermore, providing said pellets with additional coatings is also known. Sugar crystals or pellets of sucrose/starch, for example, are used as starter cores. Starter cores made from other materials, such as silicon dioxide, have also been proposed. The properties of the known starter cores are not always satisfactory, however. In particular, the existing starter cores have the disadvantage that, on contact with aqueous media, at best they disintegrate slowly and are therefore completely unsuitable for some dosage forms. Thus, dosage forms for which it is important that the pellets quickly disintegrate completely cannot be prepared with conventional starter cores. The other properties of starter cores also often leave something to be desired. Thus, conventional pellets often display large deviations from an ideal spherical form. This makes it more difficult to apply layers of active substances and other coatings.

There is therefore a need to develop new starter pellets (neutral pellets, or pellets for short) with improved properties. In particular, starter pellets should be prepared that are spherical and are suitable for further processing to oral dosage forms, in particular those with rapid release of the active substance.

This also has to be viewed against the background that the oral administration of solid dosage forms, such as tablets or capsules, is often a problem, especially with children and the elderly, as such preparations are not easy to swallow. This quite often leads to inadequate patient compliance. This applies in particular to patients who have difficulty swallowing owing to the nature of the illness. Furthermore, tablets and capsules must be taken with a sufficiently large amount of liquid, in order to ensure that the solid pharmaceutical form reaches the stomach. Patients do not always comply with this. The ingestion of a conventional preparation, such as a capsule or tablet, without a sufficient amount of liquid can, however, cause damage to the esophagus. Finally, patients wanting to take a medicine against acute complaints, such as motion sickness, headaches or allergic complaints, do not always have water or a suitable drink available.

There is therefore a need for the development of pharmaceutical forms that can also be taken easily by patients who cannot readily swallow ordinary tablets or capsules, and in particular can also be taken without liquid.

Furthermore, with conventional preparations release of the active substance is often delayed. Although in some cases this is in fact desirable, delayed release can also be a disadvantage, in particular in the case of preparations for which release of the active substance should already take place in the mouth, such as with preparations for sublingual use. There is therefore also a demand for preparations from which the active substance is released quickly, in particular even when taken without liquid.

The prior art contains various proposals for such dosage forms, but they always have disadvantages. For example, WO 98/06385 A1 describes an easily swallowed oral pharmaceutical form, comprising one or more coated particles, and more precisely defined requirements have to be imposed on the condition of the coating layers. As a result, the composition and production of the pharmaceutical forms according to this document are necessarily relatively complicated.

Furthermore, effervescent preparations for medicinal products have been proposed in the prior art. For example, WO 99/59553 A1 describes effervescent preparations that contain an effervescent mixture of a CO₂ donor and a CO₂-releasing acidic component, an active substance and other excipients. This composition necessitates complicated manufacturing stages. Furthermore, effervescent preparations often have disadvantages with respect to storage stability.

The aim of the present invention is to provide an oral pharmaceutical preparation that can be taken easily and in particular also without liquid, while avoiding the disadvantages described above.

A further aim of the invention is to provide an oral pharmaceutical preparation that releases the active substance immediately after ingestion.

Furthermore, the invention also aims to provide a method of production for a pharmaceutical preparation.

According to the invention, neutral pellets are also to be provided, which can be used for the production of pharmaceutical preparations, in particular by applying one or more layers, at least one of which contains an active substance.

To achieve the aforementioned aims, according to the invention a pharmaceutical pellet is prepared with a length/width ratio of less than about 1.4, which comprises a core that contains a water-soluble excipient and an active substance homogeneously distributed therein.

Furthermore, according to the invention, a pellet with a length/width ratio of less than about 1.4 is prepared, which contains a water-soluble excipient, and said pellet disintegrates into smaller fragments with visually discernible loss of pellet structure when the following test is carried out: (a) 200 to 800 mg of pellets is put in a Petri dish with 0.5 ml demineralized water; (b) immediately thereafter, the Petri dish is secured to the measuring cylinder holder of a tamped-volume measuring instrument; (c) after 10 seconds, 30 tamping operations are carried out; (d) immediately thereafter, the pellets are photographed.

Furthermore, according to the invention, a method is provided for the production of a pharmaceutical pellet of the type stated above, which comprises the following stages:

(a) preparation of a powdered starting material;

(b) pelletization of the powdered starting material using a pharmaceutically acceptable liquid diluent;

(c) drying of the pellet obtained.

According to a further aspect of the invention, a pharmaceutical pellet is prepared, which comprises a pellet, as mentioned above and described in more detail below, as core and one or more layers applied on the core, at least one of which contains an active substance.

The invention is described in more detail below, referring to the drawing, which illustrates the disintegration behavior of various pelletized products. For this, in each case a specified quantity of pellets was put in a Petri dish with 0.5 ml demineralized water. Immediately thereafter, the Petri dish was secured to the measuring cylinder holder of a tamped-volume measuring instrument. After 10 seconds, 30 tamping operations were carried out. Immediately thereafter, the pellets were photographed. The results are presented in the diagrams.

FIG. 1 shows the disintegration behavior of pellets according to the invention prepared from mannitol with a particle size of the pellets from 250 to 500 μm, using 400 mg of pellets for the test.

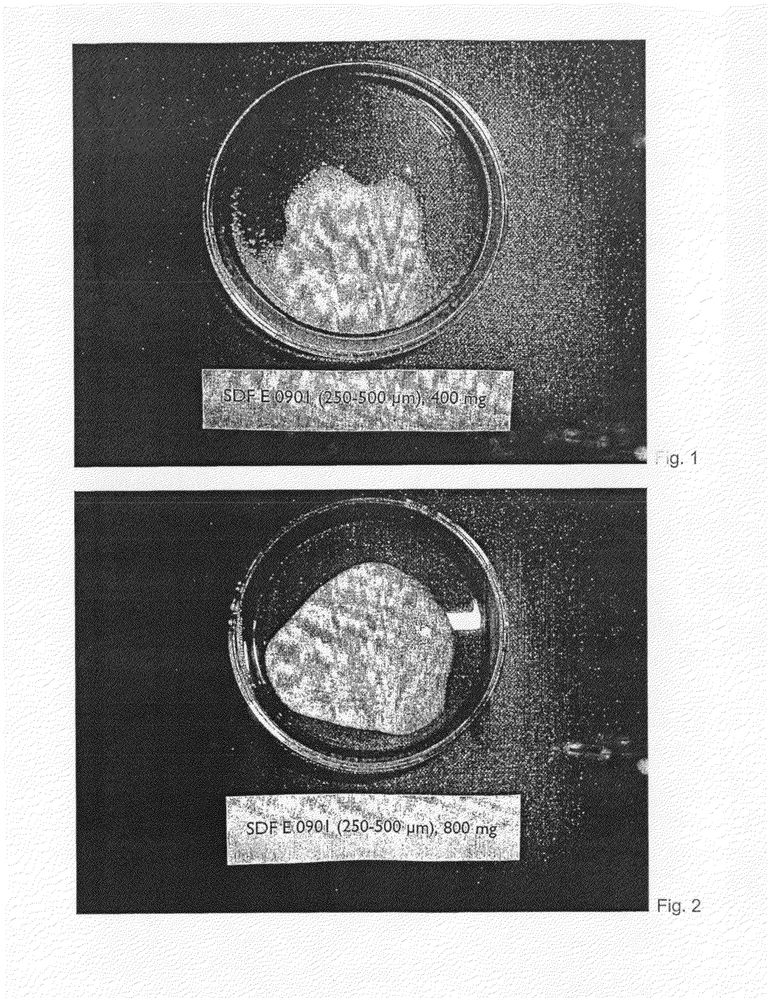

FIG. 2 shows the disintegration behavior of pellets according to the invention prepared from mannitol with a particle size of the pellets from 250 to 500 μm, using 800 mg of pellets for the test.

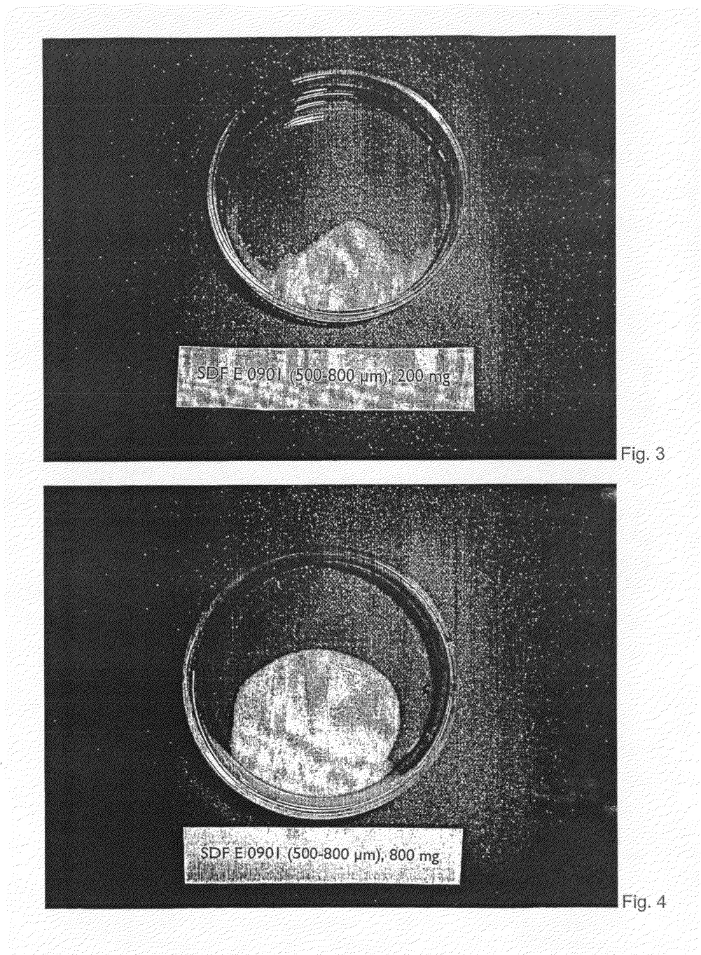

FIG. 3 shows the disintegration behavior of pellets according to the invention prepared from mannitol with a particle size of the pellets from 500 to 800 μm, using 200 mg of pellets for the test,

FIG. 4 shows the disintegration behavior of pellets according to the invention prepared from mannitol with a particle size of the pellets from 500 to 800 μm, using 800 mg of pellets for the test.

FIG. 5 shows the disintegration behavior of pellets according to the invention prepared from mannitol with a particle size of the pellets from 800 to 1000 μm, using 400 mg of pellets for the test.

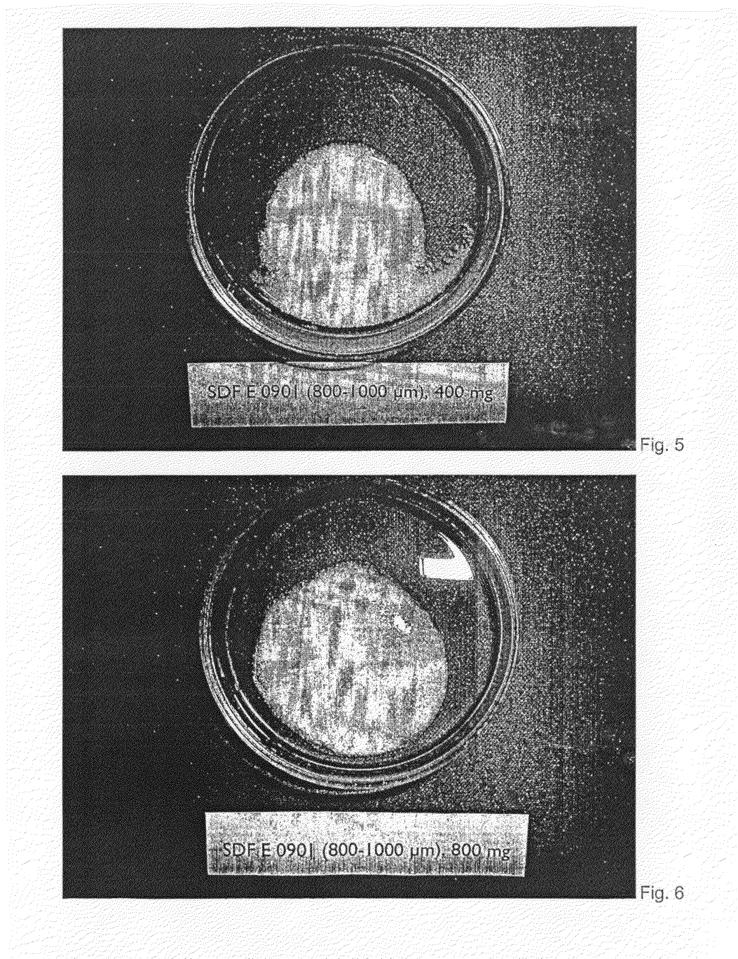

FIG. 6 shows the disintegration behavior of pellets according to the invention prepared from mannitol with a particle size of the pellets from 800 to 1000 μm, using 800 mg of pellets for the test.

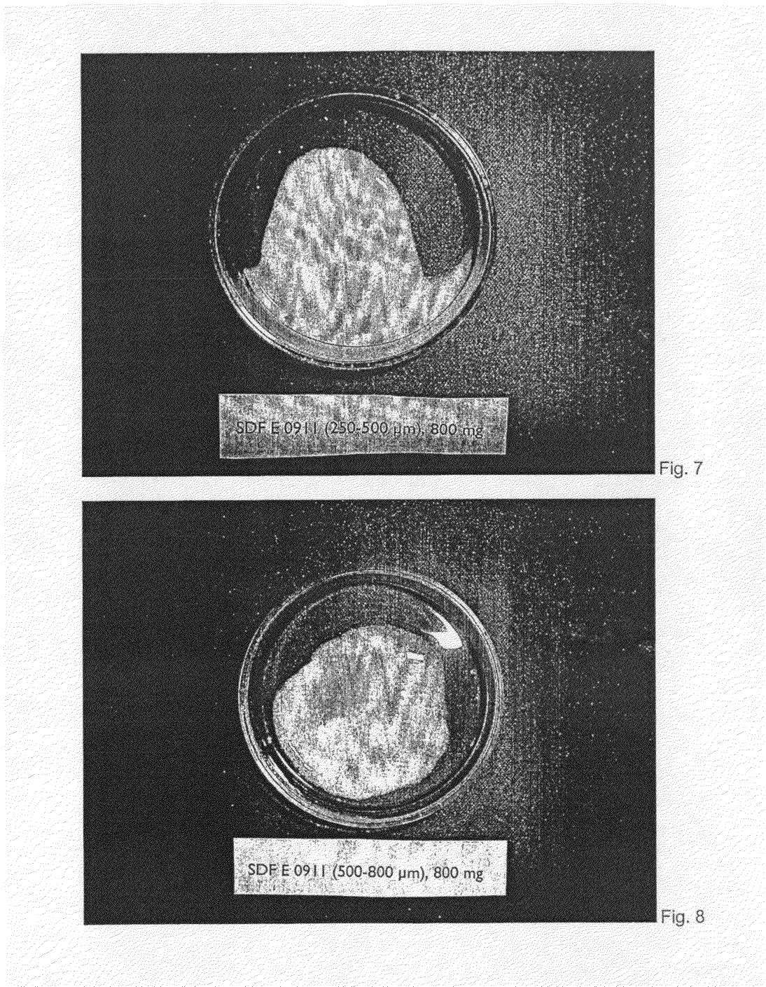

FIG. 7 shows the disintegration behavior of pellets according to the invention prepared from mannitol and polyvinylpyrrolidone (PVP) (1.5 wt. % PVP, based on the dry weight of all components), where the particle size of the pellets was 250 to 500 μm and 800 mg of pellets was used for the test.

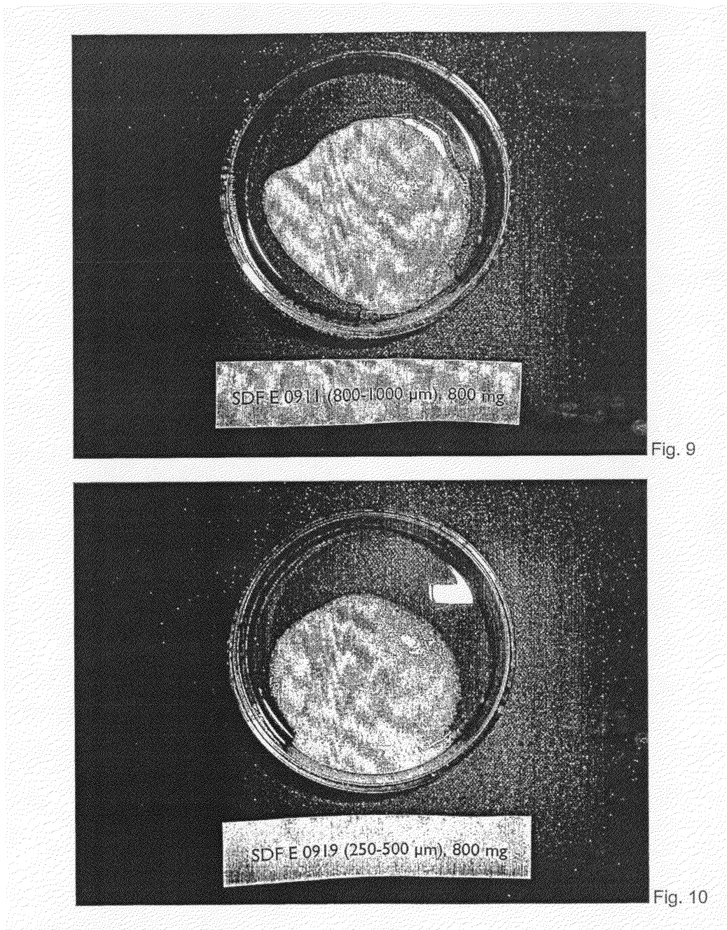

FIG. 8 shows the disintegration behavior of pellets according to the invention prepared from mannitol and PVP (1.5 wt. % PVP, based on the dry weight of all components), where the particle size of the pellets was 500 to 800 μm and 800 mg of pellets was used for the test.

FIG. 9 shows the disintegration behavior of pellets according to the invention prepared from mannitol and PVP (1.5 wt. % PVP, based on the dry weight of all components), where the particle size of the pellets was 800 to 1000 μm and 800 mg of pellets was used for the test.

FIG. 10 shows the disintegration behavior of pellets according to the invention prepared from mannitol, caffeine (7.5 wt. %, based on the dry weight of all components) and PVP (1.0 wt. %, based on the dry weight of all components), where the particle size of the pellets was 250 to 500 μm and 800 mg of pellets was used for the test.

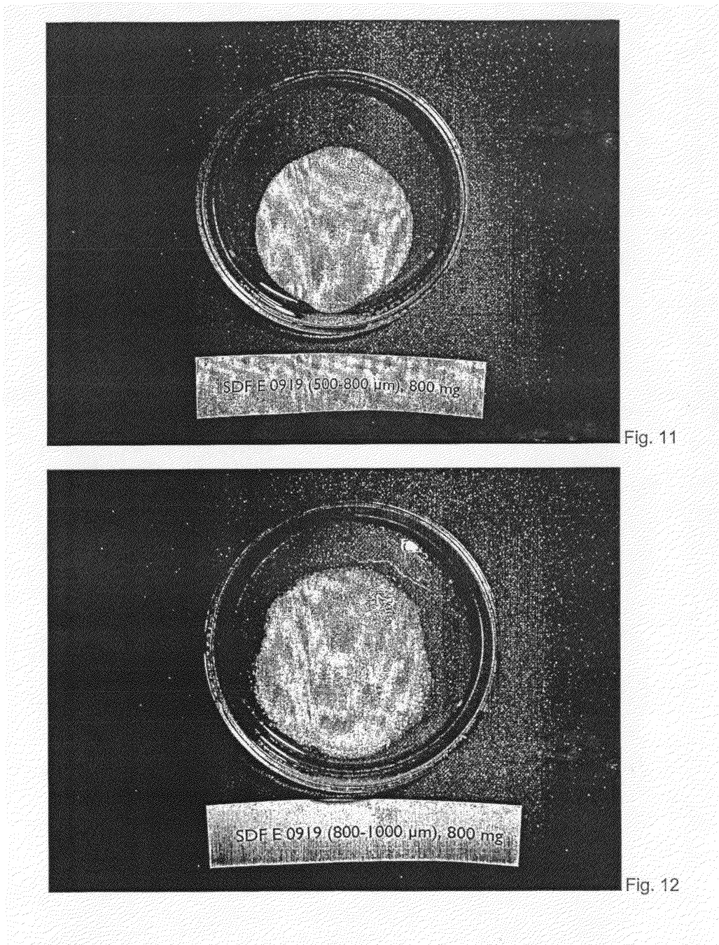

FIG. 11 shows the disintegration behavior of pellets according to the invention prepared from mannitol, caffeine (7.5 wt. %, based on the dry weight of all components) and PVP (1.0 wt. %, based on the dry weight of all components), where the particle size of the pellets was 500 to 800 μm and 800 mg of pellets was used for the test.

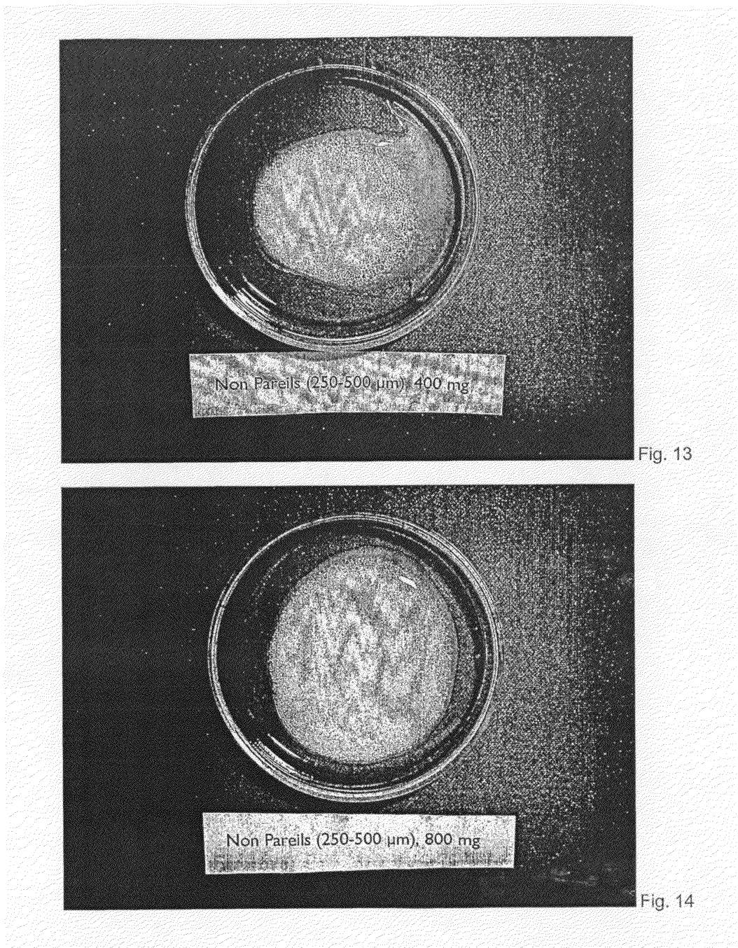

FIG. 12 shows the disintegration behavior of pellets according to the invention prepared from mannitol, caffeine (7.5 wt. %, based on the dry weight of all components) and PVP (1.0 wt. %, based on the dry weight of all components), where the particle size of the pellets was 800 to 1000 μm and 800 mg of pellets was used for the test.

FIG. 13 shows, for comparative purposes, the behavior of commercial nonpareils with a particle size from 250 to 500 μm, using 400 mg of nonpareils for the test.

FIG. 14 shows, for comparative purposes, the behavior of commercial nonpareils with a particle size from 250 to 500 μm, using 800 mg of nonpareils for the test.

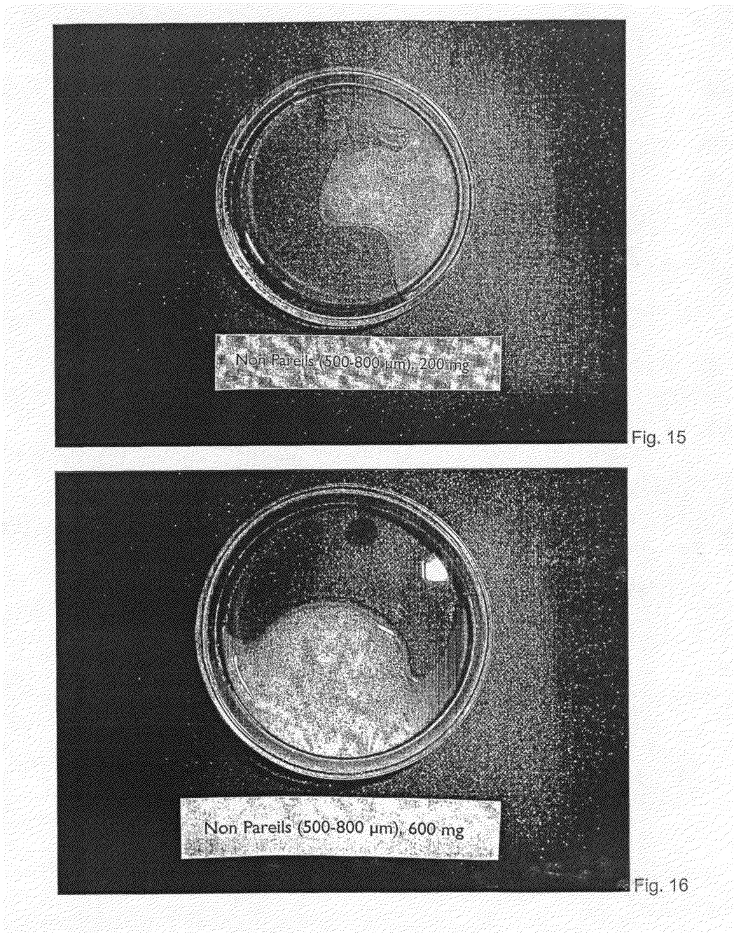

FIG. 15 shows, for comparative purposes, the behavior of commercial nonpareils with a particle size from 500 to 800 μm, using 200 mg of nonpareils for the test.

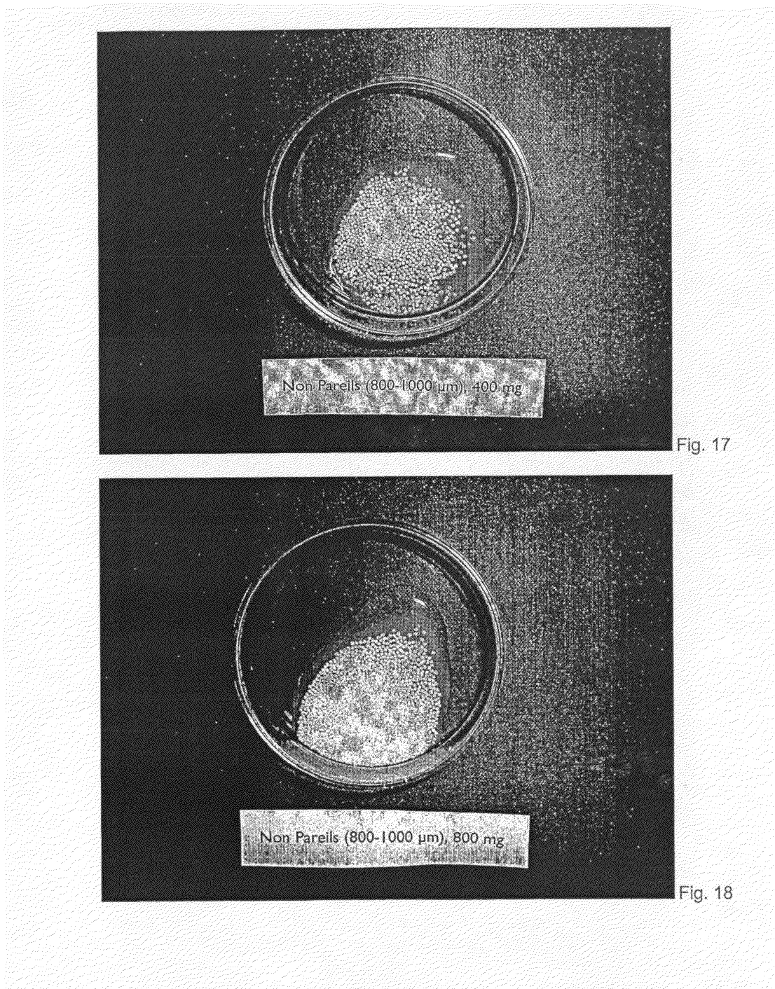

FIG. 16 shows, for comparative purposes, the behavior of commercial nonpareils with a particle size from 500 to 800 μm, using 600 mg of nonpareils for the test.

FIG. 17 shows, for comparative purposes, the behavior of commercial nonpareils with a particle size from 800 to 1000 μm, using 400 mg of nonpareils for the test.

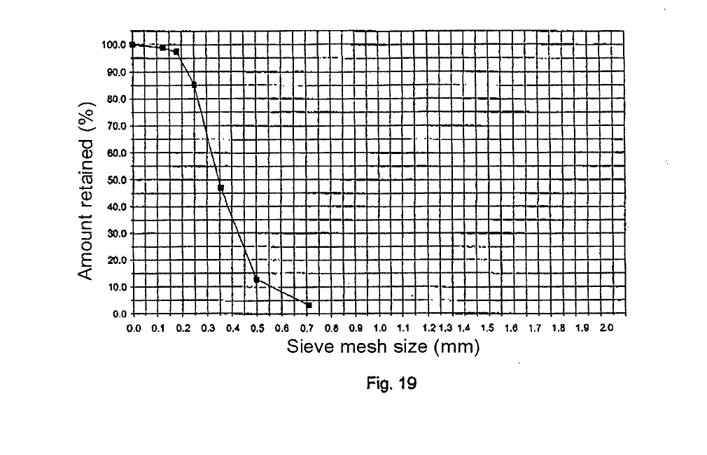

FIG. 18 shows, for comparative purposes, the behavior of commercial nonpareils with a particle size from 800 to 1000 μm, using 800 mg of nonpareils for the test.

FIG. 19 shows the particle size distribution of pellets according to the invention, which contain mannitol, crospovidone and citric acid.

Pharmaceutical pellets, which contain an active substance, and neutral pellets (starter pellets), which do not contain an active substance, are prepared according to the invention.

The pellet according to the invention is a spherical particle with a length/width ratio (i.e. a ratio of the length (largest dimension) of the pellet, divided by the width (smallest dimension), determined at an angle of 90° to the length), of less than about 1.4, preferably less than about 1.3, more preferably less than about 1.2, even more preferably less than about 1.1 and in particular less than about 1.05.

The pellet typically has a size in the range from 0.01 to 5 mm. Preferably the size is in the range from 0.1 to 1 mm, in particular in the range from 0.1 to 0.6 mm. An especially preferred range is 0.2 to 0.6 mm. A range from 0.3 to 0.5 mm is even more preferred.

The core of the pellet contains a water-soluble excipient. Water-soluble carbohydrates are especially suitable. The following may be mentioned as examples: dextran, dextrin, dextrose (glucose), fructose, lactose, maltodextrin, mannitol, sucrose, sorbitol and xylitol. Sugar alcohols are preferred. Preferably, a nonhygroscopic excipient is used. Mannitol and mixtures of mannitol and one or more of the above-mentioned excipients are especially preferred. Mannitol is quite especially preferred.

There is no particular restriction on the amount of excipient in the pellet according to the invention. Typically it is 50 parts by weight or more, in particular 70 parts by weight or more, based on the dry weight of all constituents of the core.

Compositions according to the invention are basically suitable for the administration of any biologically active substances, such as therapeutically and/or prophylactically effective substances (active substances) that can be administered orally. Examples of classes of active substances are analgesics, COX-2 inhibitors, antacids, antiasthmatics, broncholytics, antibiotics, psychoactive drugs, antirheumatics, antidiabetics, antiallergics, antihistamines, antihypotensives, antitussives, antihypertensives, laxatives, mucolytics, expectorants, H₂ blockers, local anesthetics, antiemetics, prokinetics, antilipemics, antimigraine drugs, sympathomimetics, vitamins and minerals. Preferred classes of active substances are analgesics, antimigraine drugs, antiallergic agents, psychoactive drugs and vitamins and minerals.

Examples of analgesics are ibuprofen, ketoprofen, paracetamol and acetylsalicylic acid. Examples of COX-2 inhibitors are nimesulide, meloxicam, naproxen, propyphenazone and metamizole. Examples of antacids are hydrotalcite, magaldrate and calcium carbonate. Examples of antiasthmatics and broncholytics are salbutamol, tulobuterol, terbutaline, cromoglycic acid, ketotifen and theophylline. Examples of antibiotics are quinolones, tetracyclines, cephalosporins, penicillins, macrolides, sulfonamides and polypeptides.

Examples of psychoactive drugs are benzodiazepines, haloperidol, amitriptyline and carbamazepine. Examples of antirheumatics are phenylbutazone, indometacin, diclofenac and piroxicam. Examples of antidiabetics are metformin, glibenclamide, acarbose and glisoxepide. Examples of antiallergics and antihistamines are astemizole, terfenadine, loratadine, clemastine, bamipine and cetirizine. Examples of antihypotensives are etilefrine, norfenefrine and dihydroergotamine mesylate. Examples of antitussives are codeine, dextromethorphan, clobutinol and dropropizine. Examples of antihypertensives are beta blockers, such as propranolol, atenolol, metoprolol and prazosin and calcium antagonists, such as nifedipine, nitrendipine, diltiazem, verapamil, felodipine and nimodipine. Examples of laxatives are sodium picosulfate, lactulose and lactitol. Examples of mucolytics and expectorants are ambroxol, bromhexine, guaifenesin, acetylcysteine and carbocisteine. Examples of H₂ blockers are ranitidine, famotidine and pirenzepine. Examples of local anesthetics are benzocaine, lidocaine and procaine. Examples of antiemetics and prokinetics are metoclopramide, domperidone, meclozine and dimenhydrinate. Examples of antilipemics are fenofibrate, bezafibrate, pravastatin and fluvastatin. Examples of antimigraine drugs are caffeine, dihydroergotamine, ergotamine, sumatriptan and pizotifen. Examples of sympathomimetics are pseudoephedrine and pholedrine.

A preferred group of active substances comprises zolmitriptan, acetylsalicylic acid, rizatriptan, risperidone, selegiline, olanzapine, cetirizine, ondansetron, loperamide, buprenorphine, mirtazapine, apomorphine and piroxicam.

Pharmaceutically compatible salts or esters of active substances can also be used according to the invention.

According to a preferred embodiment, a derivative having good solubility in water (salt or ester) of an active substance is used.

The pellets according to the invention offer particular advantages in the case of low-dose active substances, sublingually bioavailable active substances, active substances desired to have rapid onset of action and active substances for which improvement of patient compliance is important.

According to the invention, the active substance can also be a combination of two or more substances, which preferably are selected from the aforementioned agents.

There is no particular restriction on the amount of active substance in the pharmaceutical pellet according to the invention. Typically the pellet contains 50 parts by weight or less and preferably 30 parts by weight or less of active substance, based on the dry weight of all constituents of the core as 100 parts by weight. It is especially preferable for the content of active substance to be between 1 and 20 parts by weight, for example between 3 and 10 parts by weight, on the basis stated above.

As already explained, the pellets according to the invention comprise a core, which contains a water-soluble excipient and an active substance homogeneously distributed therein. According to a preferred embodiment the core consists of the two stated constituents, concrete examples of which were given above.

According to a further preferred embodiment the core contains as a further constituent at least one water-soluble binder. The water-soluble binder is preferably selected from the group comprising calcium carboxymethylcellulose, polymers based on acrylic acid (carbopol), gelatin, hydroxyethylcellulose, hydroxypropylcellulose, hydroxypropyl methylcellulose, polyethylene glycol (macrogol), methylcellulose, sodium carboxymethylcellulose, sodium carboxymethyl starch, polyoxypropylene-polyoxyethylene block polymers (poloxamers), polyvinyl alcohol, polyvinylpyrrolidone (povidone) and starch. Preferred binders include gelatin, sodium carboxymethylcellulose and polyvinylpyrrolidone (povidone). Polyvinylpyrrolidone is quite especially preferred. Polyvinylpyrrolidone (povidone) is commercially available in suitable form, for example as Collidon 30.

If the pellet core contains binder, the amount is preferably not more than 20 parts by weight, in particular not more than 10 parts by weight, more preferably not more than 5 parts by weight, especially not more than 2 parts by weight and quite especially preferably not more than 1 part by weight, based on the dry weight of all constituents of the core as 100 parts by weight. Preferred ranges for the amount of binder are 0.5 to 20 parts by weight, in particular 0.5 to 10 parts by weight, more preferably 0.5 to 5 parts by weight, especially 0.5 to 2 parts by weight and quite especially preferably 0.5 to 1 part by weight.

According to another preferred embodiment the pellets contain an excipient that is insoluble but swellable in water. Examples are

Crospovidone

Alginic acid

Calcium sulfate

Carboxymethylcellulose calcium

Carboxymethylcellulose sodium

Microcrystalline cellulose

Pulverized cellulose

Croscarmellose sodium

Modified cellulose gum

Guar gum

Hydroxypropylcellulose

Methylcellulose

Sodium alginate

Sodium starch glycolate

Starch (maize, potato, rice, tapioca, wheat) Pregelatinized starch, pregelatinized maize starch

Amylose

Amylopectin starch

Pectin

Agar

Xanthan gum

Soya polysaccharides

Xylan

Polymeric cyclodextrin

Polacrilin potassium

Bentonite

Sodium carboxymethyl starch

Preferred swellable excipients display a maximum swelling of more than 5%, in particular more than 30%, especially more than 40%, quite especially more than 60%, even more preferably more than 80% and most preferably more than 100%. Determination of maximum swelling is described by D. Gissinger and A. Stamm, A comparative evaluation of the properties of some tablet disintegrants, Drug Development and Industrial Pharmacy, 6(5), 511-536 (1980). This is expressly referred to.

Preferred substances are starch, pregelatinized starch, sodium starch glycolate, guar gum, carboxymethylcellulose sodium, croscarmellose sodium and crospovidone. Crospovidone is especially preferred.

According to the invention, it is preferable if the swellable substance is in finely divided form. Advantageously, a micronized material can be used.

The amount of the swellable excipient is in the range from 0.5 to 30 wt. %, based on the total amount of the ingredients of the pellet. The optimal amount is determined by tests. For example, an amount from 5 to 15 wt. % is suitable. An amount in the range from 5 to 25 wt. % is advantageous, in particular more than 10 to 25 wt. %, such as more than 10 to 15 wt. %.

It was found, according to the invention, that the swellable excipient improves the execution of the process. It is easier to achieve a specified narrow particle size distribution. The properties of the pellets are not impaired.

The core can contain other optional constituents in addition to the stated constituents. Both water-soluble and water-insoluble excipients can be contained additionally. These include agents for improving taste, aromatics, colorants, preservatives, physical stabilizers and chemical stabilizers, such as acid, basic or buffering components. The optional constituents also include antiadherents, disintegration accelerators and disintegration inhibitors. The amount of the further optional constituents is typically not more than 30 parts by weight, based on the dry weight of all constituents of the core as 100 parts by weight. Preferably the amount of the further optional constituents is not more than 10 parts by weight.

According to a further preferred embodiment, pellets are prepared that contain, in the core, 100 parts by weight of water-soluble excipient, such as a water-soluble carbohydrate, in particular mannitol, 1 to 10 parts by weight and in particular 3 to 10 parts by weight of active substance, 0.5 to 20 parts by weight, especially 0.5 to 10 parts by weight, more preferably 0.5 to 5 parts by weight, in particular 0.5 to 2 parts by weight and quite especially preferably 0.5 to 1 part by weight of water-soluble binder and 0 to 10 parts by weight of other excipients and preferably consist thereof.

According to a further preferred embodiment, pellets are prepared that contain, in the core, 100 parts by weight of water-soluble excipient, such as a water-soluble carbohydrate, in particular mannitol, 0.5 to 20 parts by weight, especially 0.5 to 10 parts by weight, more preferably 0.5 to 5 parts by weight, in particular 0.5 to 2 parts by weight and quite especially preferably 0.5 to 1 part by weight of water-soluble binder, and 0 to 10 parts by weight of other excipients and preferably consist thereof.

According to a further preferred embodiment, pellets are prepared that contain, in the core, 100 parts by weight of water-soluble excipient, such as a water-soluble carbohydrate, in particular mannitol, 1 to 10 parts by weight and in particular 3 to 10 parts by weight of active substance, 5 to 35 parts by weight, preferably 5 to 25 parts by weight and in particular more than 10 to 25 parts by weight of a swellable excipient, in particular with the swelling behavior defined above, especially crospovidone, and 0 to 10 parts by weight of other excipients and preferably consist thereof.

According to a further preferred embodiment, pellets are prepared that contain, in the core, 100 parts by weight of water-soluble excipient, such as a water-soluble carbohydrate, in particular mannitol, 5 to 35 parts by weight, preferably 5 to 25 parts by weight and in particular more than 10 to 25 part by weight of a swellable excipient, in particular with the swelling behavior defined above, especially crospovidone, and 0 to 10 parts by weight of other excipients and preferably consist thereof.

According to a preferred embodiment, the pellet according to the invention consists of the core defined above.

According to a further embodiment, the pellet according to the invention has a core of the type described above and, applied on the core, one or more coatings. Water-soluble coatings are preferred. A coating can serve as a protective layer.

A method for the production of pharmaceutical pellets is also provided according to the invention. The method comprises, in a first stage, the preparation of a powdered starting material. The powdered starting material comprises a water-soluble excipient. The powdered starting material is preferably submitted to a treatment, for example in a shearing mixer, in order to break up aggregates. The powdered starting material can also be a mixture of a water-soluble excipient and one or more further ingredients of the pellets being produced, for example binders. In this case the preparation of the powdered starting material typically includes mixing of the components. For example, a water-soluble excipient and an active substance are mixed together.

Preferably, a homogeneous mixture of the constituents is achieved. This too can take place in a shearing mixer.

Preferably, a powdered starting material of restricted grain size is used. In particular the powdered starting material comprises an excipient whose grain size is restricted. Furthermore, a narrow grain size distribution is preferred for the powdered starting material and in particular for the excipient.

For example, in the production of pellets with a size of 300 to 500 μm it has proved beneficial to use a powdered starting material, in particular an excipient, especially mannitol, for which the sieving residue at a sieve mesh size of 160 μm is less than 10 wt. % and in particular less than 5 wt. %. Moreover, powdered starting materials, in particular a powdered excipient, such as in particular mannitol, are preferred for which the sieving residue at a sieve mesh size of 40 μm is in the range from 50 to 80 wt. %. Especially preferably, the sieving residue, at a mesh size of 160 μm, is in the range from 1 to 5 wt. % and, at a mesh size of 40 μm, in the range from 50 to 70 wt. %.

The powdered starting material can be moistened before the pelletization stage. This is done by adding a pharmaceutically acceptable diluent. This can be the same diluent as will be used in the subsequent pelletizing stage, or it can be a diluent of a different composition. The diluent can be an organic liquid. Preferably it is water or an aqueous solution or dispersion. The liquid can contain, as constituents, a binder and/or an active substance and/or further ingredients of the core. The amount of the pharmaceutically acceptable diluent is preferably such that a wetted powdered starting material is achieved, the amount of liquid added being below the amount that is necessary for formation of granule-type structures. Preferably, uniform moistening of the powdered starting material is provided. This can take place using a suitable mixer, such as a shearing mixer.

If several pulverulent components are used, mixing and preliminary moistening can take place in one step, for example in a high-speed mixer.

In the pelletizing stage, pellets are formed from the optionally premoistened starting material with addition of a pharmaceutically acceptable liquid diluent. The properties of the diluent must satisfy the same criteria as for the diluent used for preliminary moistening.

The diluent can contain an active substance.

It is also possible to introduce pulverulent constituents, for example a pulverulent active substance, during pelletization, if the process is conducted in such a way that homogeneous, thorough mixing is achieved. As the pellets according to the invention contain an active substance, this must either be present in the powdered starting material or must be introduced during pelletization as a constituent of the diluent or in powder form. Combinations of these measures are also possible.

According to a preferred embodiment, the method comprises the following stages:

(a) preparation of a powdered starting material, which includes a water-soluble excipient;

(b) feed of the powdered starting material, which optionally is wetted with a pharmaceutically suitable liquid diluent, into a device, which has:

a rotating chamber with a cylindrical wall extending axially,

a device for conveying air through the rotating chamber from the bottom,

a spraying device for feeding liquid into the chamber,

one or more inlets for introducing the powdered mixture,

a rotor, which rotates about a vertical axis of rotation, wherein the rotor is arranged in the rotating chamber, has a central horizontal surface and in at least the outermost third of the rotor has the form of a conical generated surface with a slope directed outward and upward between 10° and 80°, where the conical generated surface has a circular upper edge, which lies in a plane that is perpendicular to the axis of rotation,

a plurality of guide vanes each with an outer end secured statically to the cylindrical wall of the rotating chamber above the plane formed by the upper edge of the conical generated surface of the rotor, and an inner end that extends into the rotating chamber and is arranged tangentially to the cylindrical wall of the rotating chamber and, perpendicularly to the axis of rotation, essentially has the form of a circular arc or a spiral,

(c) rotation of the rotor, so that the product is caused to circulate by kinetic energy for a sufficient period of time, moves from the rotor to the inner surface of the guide vanes, before it drops back onto the rotor, while optionally air is fed and/or a pharmaceutically acceptable liquid is sprayed into the rotating chamber, so that solid pellets with a desired diameter are formed.

A suitable device is described in DE 197 50 042 A1. Further details on the construction of the device and its operation are described in the examples presented below and the reference examples.

During pellet formation, a pharmaceutically acceptable diluent is supplied, as was described above. The amount is selected in particular in relation to the components of the starting material, the desired pellet size and the other operating variables, for example the amount of air supplied. A suitable amount can be determined by routine tests by a person skilled in the art on the basis of the examples presented here and the reference examples.

The pellets finally obtained are dried. If desired, they can then also be provided with one or more coatings.

In particular, neutral pellets according to the invention of the type described above can be used for the production of a pharmaceutical preparation. For this, the neutral pellets can be provided with coatings in a known manner. Accordingly, according to the invention, a pharmaceutical pellet is also provided that comprises a neutral pellet according to the invention as core and one or more layers applied on the core, at least one of which contains an active substance.

Layers containing active substance can be applied in any manner. For example, a solution of an active substance, which optionally also contains excipients such as binders, can be sprayed on. It is also possible to use a suspension instead of a solution. Application of a pulverulent active substance is also possible. For this, neutral pellets are contacted in a suitable manner with a powdered active substance, with addition of a solvent and/or a binder.

Compositions according to the invention, which can be prepared using the neutral pellets, are suitable basically for the administration of any biologically active substances, such as substances having therapeutic and/or prophylactic effects (active substances) that can be administered orally. Reference should be made to the groups of active substances already mentioned and to the specific examples of active substances, also already mentioned.

The pellets obtained according to the invention can find application as a pelletized product, which comprises a plurality of pellets. A pelletized product comprises a collection of pellets, typically 50 or more, preferably 100 or more pellets. A pelletized product according to the invention comprises primarily pellets that fulfill the criteria according to the invention. Preferably at least 90%, in particular at least 95% and quite especially preferably at least 98% of the pellets have a length/width ratio of less than about 1.4, preferably less than about 1.3, more preferably less than about 1.2, even more preferably less than about 1.1 and in particular less than about 1.05. The pellets that have the preferred length/width ratio also preferably display the other requirements on pellets according to the invention, in particular also the requirements that are specified in the claims or are listed as preferred in the present description.

The quantity of pellets in a pelletized product according to the invention can also be specified by the weight. A pelletized product can for example contain 50 to 2000 mg, preferably 100 to 1000 mg and in particular 200 to 800 mg of pellets.

The size of the pellets in a pelletized product according to the invention can for example be in the range from 250 to 1000 μm.

According to the invention, a pelletized product with a narrow particle size distribution of the pellets contained is preferred. Preferably at least 90 wt. %, in particular at least 95 wt. % of the pellets in the pelletized product have a diameter that is within a range of ±30%, preferably ±20% and in particular ±10% of the mean diameter of all the pellets.

For preparation of a pelletized product, a suitable quantity of pellets can for example be packed in a bag. The pelletized product can also contain other components in addition to the pellets according to the invention.

The preparation can be made available to patients in this packaged form. The patients can remove the pelletized product from the bag and take it without liquid. Alternatively the patients can take the product with a drink or put it in a drink and ingest the resultant solution/suspension.

Another alternative is to prepare a tablet from the pellets by compaction. For example, pellets can be compressed, optionally together with one or more excipients, such as glidants. For production of a tablet it is also possible to employ a pelletized product according to the invention. The tablet can be used for oral and also in particular for sublingual administration of an active substance.

If pellets according to the invention, in particular pellets with mannitol as excipient, are taken without liquid, they disintegrate on the tongue into smaller, very soft particles and then dissolve within a few seconds. The sensation during melting until completely dissolved can be described as pleasant. Owing to the soft consistency of the pellets in the mouth and rapid dissolution, there is no sensation of a foreign body in the mouth.

The rapid disintegration of pellets according to the invention can be ascertained visually. For this, the following test is carried out: (a) 200 to 800 mg of pellets is put in a Petri dish with 0.5 ml demineralized water, (b) immediately thereafter, the Petri dish is secured to the measuring cylinder holder of a tamped-volume measuring instrument, (c) after 10 seconds, 30 tamping operations are carried out, (d) immediately thereafter, the pellets are photographed.

With pellets according to the invention, loss of pellet structure and disintegration into small fragments can be ascertained visually.

For a pelletized product that contains a plurality of pellets corresponding to the above definition, it is also possible to measure the disintegration time. This is performed in a disintegration tester with a perforated bottom. This is used for testing the disintegration time of the pellets in demineralized water at 37° C. The amount of water is chosen such that the pellets are just completely covered. The quantity of pellets is preferably 500 mg. The time until disintegration or dissolution of the pellets is measured.

With a pelletized product according to the invention, a disintegration time of less than 1:30 min, preferably less than 1:00 min, is measured.

Pellet products can also be assessed organoleptically. The assessment is carried out by testers. Preferably at least 10 testers are employed. The following possibilities are predefined for classification of the product by a tester:

0=no sensation of a foreign body

1=paste-like, soft

2=sandy

3=gritty

The assessment that is assigned to a pelletized product corresponds to that given by the majority of the testers.

Preferably the testers are given the opportunity, for comparison purposes, to test a pelletized product that has a long disintegration time, in particular of more than 1:30 min. Commercial nonpareils are such a product. The pellets of the comparative product typically have a size in the same range as the pellets of the product to be assessed.

For the organoleptic test, typically 200 mg of pellets is used. After assessing a sample, the testers are always given the opportunity to rinse their mouths with water.

Assessment according to the classification given above takes place at fixed time intervals after taking the pelletized product. Especially suitable time intervals are 5, and/or 20 seconds.

In the test described above, a pelletized product according to the invention is assessed at most as 2=sandy by the majority of at least 10 testers, in assessment after 5 seconds. A pelletized product according to the invention is preferably not classified as gritty by the testers. Furthermore, a pelletized product according to the invention is preferably classified at most as 1=paste-like, soft by the majority of at least 10 testers, in assessment after 10 seconds. Moreover, it is preferable if a pelletized product according to the invention no longer causes any sensation of a foreign body in the mouth (classification=0) for the majority of at least 10 testers, in assessment after 20 seconds.

### EXAMPLE 1

The present example describes the production of pellets from mannitol, which do not contain an active substance. For a person skilled in the art, however, it is immediately apparent that with appropriate application of the method described, for example to a composition that also contains an active substance in addition to mannitol, pellets containing an active substance can be produced.

Put 1250 g mannitol 60 in a shearing mixer (type Glatt VG-10). Run the apparatus for 2 minutes, in order to break up aggregates. The settings are: chopper (2000 rev/min); impeller (500 rev/min). Then add 200 g demineralized water within 5 minutes, operating the shearing mixer at the settings stated above, to obtain a uniformly moistened material. The amount of liquid added is less than the amount that is necessary for the formation of granule-type structures (drying loss approximately 13%; the drying loss can be determined experimentally using an infrared balance).

Next, pellets are prepared from the moistened mannitol powder. For this, the moistened mannitol powder is transferred to a device as described previously. More precisely, the moistened material is put in a device in which a rotor with conical generated surface directs the pre-wetted material in a horizontal product stream up to the edge of the rotating vessel. The centrifugal force presses the product against the wall (main product stream). In the region of the air gap between rotor and wall (stator), the tumbling product moves upward (secondary product stream). Fixed guide vanes on the wall force the tumbling bed in the middle of the rotor back downward. The product stream can be described as a spiral, rotating stream.

The rotary speed of the rotor (which affects the friction), the slope of the conical generated surface of the rotor, the number of guide vanes and the shape of the guide vanes influence the product stream and hence also the shape and density of the pellets formed.

For example, pellets can be produced using a rotor with a slope of the generated surface of 30° or of 45° using two or four flat or two steep guide vanes. An example of rotor diameter is 29 cm. A rotor of this kind was used for the examples given below. A suitable rotor speed is in the range from 600 to 1200 rev/min.

In the production method being explained, in addition a pharmaceutically acceptable liquid, in particular water or an aqueous solution of a binder, is sprayed directly onto the tumbling bed. This is done for example using a centrifugal atomizer. A suitable amount of liquid is in the range from 50 to 600 g, and a suitable spraying rate is in the range from 10 to 30 g/min.

The amount of liquid supplied during preliminary moistening and during pelletization has an influence on the process and on the product properties. However, the moisture content also depends on the volume of air supplied, the inlet temperature of the air and its humidity. On the basis of the parameters given here as examples, a person skilled in the art can find suitable parameters for the production of pellets in an individual case by routine tests.

Particularly good results are achieved with the following parameters: the slope of the conical generated surface of the rotor is 45°. The device is equipped with two flat guide vanes. The rotor is operated at a speed of 1000 rev/min. For pelletization, liquid is injected at a spraying rate of 20 g/min. The amount of liquid supplied is 50 to 600 g, depending on the target pellet size and the composition of the liquid (e.g. water, solution, dispersion). After completion of the spraying process, the rotor is run for a further 2 minutes at a reduced rotor speed of 600 rev/min. The mannitol pellets thus obtained are dried at 60° C., until the drying loss is less than 0.5%.

### EXAMPLE 2

The present example describes the production of mannitol pellets, containing gelatin as water-soluble binder. For a person skilled in the art it is immediately apparent that with corresponding application of the method described, for example to a composition that additionally also contains an active substance, pellets containing an active substance can be produced.

Moisten 1250 g mannitol 60 in a shearing mixer with 200 g demineralized water, as in reference example 1.

Pelletization is then carried out. A rotor is used for which the slope of the conical generated surface is 45°. The device is equipped with two flat guide vanes. The rotor is operated at a speed of 1000 rev/min. 130 g of a 5% aqueous solution of gelatin (cold-water-soluble, Solugel LB type A) is injected at a spraying rate of 20 g/min. Finally, the pellets thus obtained are dried at 37° C., until the drying loss is less than 0.2%. Based on the dry weight of the components, the pellets contain 99.5% mannitol and 0.5% gelatin.

### EXAMPLE 3

This example relates to the production of mannitol pellets containing gelatin as binder, the gelatin being added as a solid in the preliminary wetting stage. For a person skilled in the art it is immediately apparent that with corresponding application of the method described, for example to a composition that additionally also contains an active substance, pellets containing an active substance can be produced.

1247.5 g mannitol 60 and 2.5 g cold-water-soluble gelatin (Solugel LB type A) are put in a shearing mixer (type Glatt VG-10). The apparatus is operated for 2 minutes, in order to break up aggregates. The settings are: chopper (2000 rev/min); impeller (500 rev/min). Then 200 g demineralized water is added within 5 minutes, to wet the powder. The amount of liquid added should be less than the amount that is necessary for the formation of granule-type structures (drying loss approximately 13%). During addition of the liquid the shearing mixer is operated at the settings stated above, to obtain a uniformly moistened material.

Then the mixture is transferred to a device as described previously. A rotor with a conical generated surface with a slope of 45° is used. The device is equipped with two flat guide vanes. The rotor is operated at a speed of 1000 rev/min, and 86 g water is injected at a spraying rate of 20 g/min. After completion of injection, the rotor is operated at a reduced speed of 600 rev/min for a further 2 minutes. Then the pellets are dried at 37° C., until the drying loss is less than 0.2%.

### EXAMPLE 4

This example describes the production of mannitol pellets with gelatin as binder, the gelatin being added in dissolved form in the preliminary wetting stage. For a person skilled in the art it is immediately apparent that with corresponding application of the method described, for example to a composition that additionally also contains an active substance, pellets containing an active substance can be produced.

Dissolve 6.25 g cold-water-soluble gelatin (Solugel LB type A) in 200 g demineralized water. Put 1250 g mannitol 60 in a shearing mixer (type Glatt VG-10). Operate the mixer for 2 minutes, in order to break up aggregates. Then add the gelatin solution within 5 minutes, and operate the mixer to produce a wetted powder mixture.

Then transfer the wetted mixture to a device as described previously.

A rotor with a conical generated surface with a slope of 45° is used. The device is equipped with two flat guide vanes. The rotor is operated at a speed of 1000 rev/min, and 161 g water is injected at a spraying rate of 20 g/min. After completion of injection, the rotor is operated at a reduced speed of 600 rev/min for a further 2 minutes. Then the pellets are dried at 37° C., until the drying loss is less than 0.2%.

### EXAMPLE 5

This example describes the production of mannitol pellets with povidone as binder, povidone being added in the preliminary wetting stage. For a person skilled in the art it is immediately apparent that with corresponding application of the method described, for example to a composition that additionally also contains an active substance, pellets containing an active substance can be produced.

Two formulations are investigated. The first formulation (formulation A) comprises 1250 g mannitol 60 and 6.25 g Collidon 30. The second formulation (formulation B) comprises 1250 g mannitol 60 and 18.75 g Collidon 30.

In each case the povidone is dissolved in demineralized water. The mannitol is put in a shearing mixer (type Glatt VG-10). The apparatus is operated for 2 minutes. The settings are: chopper (2000 rev/min); impeller (500 rev/min).

The amount of water is in each case 200 g.

The liquid is added within 5 minutes, achieving a drying loss of about 13%. Then pellets are produced from the individual mixtures. For this, in each case the moistened mixtures are transferred to a device as described previously. The inlet air temperature is 30° C. The inlet air volume is 100 m³/h. The centrifugal atomizer is operated at 50%.

For the first batch, a rotor with a slope of the conical generated surface of 45° is used. Two flat guide vanes are provided, and the rotor speed is 1000 rev/min. 132 g liquid is injected at a spraying rate of 20 g/min.

For the second batch, a rotor with a slope of the conical generated surface of 45° is also used, and once again two flat guide vanes are provided. The rotor speed is 1000 rev/min, the amount of liquid injected is 108 g and the spraying rate is 20 g/min.

After completion of spraying, the process is continued for 2 minutes at a rotor speed of 600 rev/min.

The pellets from batch A are dried in a stove at 60° C. until the drying loss is less than 0.5%. Of the pellets from batch B, one portion is dried in a stove (60° C.), and the rest in a fluidized bed (Glatt GPCG-1). The inlet air temperature is 60° C., and the inlet air volume is 50 to 100 m³/h. A drying loss of less than 0.5% is achieved.

### EXAMPLE 6

This example relates to the production of mannitol pellets containing citric acid. Citric acid serves as a model substance for an active substance.

1187.5 g mannitol 60 and 62.5 g citric acid (anhydrous) are put in a shearing mixer (type Glatt VG-10). The apparatus is operated for 2 minutes initially. Then preliminary wetting is carried out by adding demineralized water (90.0 g) within 5 minutes. A drying loss of about 5% is achieved. In the preliminary wetting stage, the shearing mixer is operated at the following settings: chopper (2000 rev/min); impeller (500 rev/min).

Then pellets are produced from the mixture. For this, the moistened mixture of mannitol and citric acid is transferred to a device as described previously. The device has a rotor for which the slope of the conical generated surface is 45°. Two flat guide vanes are provided. The rotor speed is 1000 rev/min. 390 g liquid (demineralized water) is injected at a spraying rate of 20 g/min. The inlet air temperature is 30° C., and the inlet air volume is 100 m³/h. The centrifugal atomizer is operated at a speed of 50%. After spraying, the process is continued for a further 2 minutes at a rotor speed of 600 rev/min.

The pellets obtained are finally dried in a stove at 60° C. until the drying loss is less than 0.5%.

### EXAMPLE 7

This example relates to the production of mannitol pellets containing povidone as binder and citric acid. Citric acid serves as a model substance for an active substance.

1175.0 g mannitol and 62.5 g citric acid (anhydrous) are put in a shearing mixer (type Glatt VG-10). The apparatus is operated for 2 minutes. 12.5 g Collidon 30 is put in 110.0 g demineralized water. The Collidon solution is then added within 5 minutes to the mixture of mannitol and citric acid in the shearing mixer. The total running time of the shearing mixer is 7 minutes. A drying loss of about 6% is achieved.

The shearing mixer (type Glatt VG-10) is operated at the following settings: chopper (2000 rev/min); impeller (500 rev/min).

Then the mixture is transferred to a device as described previously. A rotor with a conical generated surface with a slope of 45° is used. The device is equipped with two flat guide vanes. The rotor is operated at a speed of 1000 rev/min, and 170 g demineralized water is injected at a spraying rate of 20 g/min. After completion of injection, the rotor is operated at a reduced speed of 600 rev/min for a further 2 minutes.

In the subsequent drying stage the pellets are dried in a fluidized bed (Strea-1) at an inlet air temperature of 60° C. and an air flow level of 5 until the drying loss is less than 0.5%. Orange flavoring (0.01 g) is dissolved in demineralized water (10 ml); 5 ml is added during the drying stage.

### EXAMPLE 8

This example relates to the production of mannitol pellets containing povidone as binder and citric acid.

1206.25 g mannitol 60 and 37.5 g citric acid (anhydrous) are put in a shearing mixer (type Glatt VG-10). The apparatus is operated for 2 minutes. 6.25 g povidone (Collidon 30) is dissolved in 120.0 g demineralized water. The solution is added within 5 minutes to the shearing mixer with the mixture of mannitol and citric acid. The total running time of the shearing mixer is 7 minutes. A drying loss of about 7% is achieved. The shearing mixer is operated at the following settings: chopper (2000 rev/min); impeller (500 rev/min).

Then the mixture is transferred to a device as described previously. A rotor with a conical generated surface with a slope of 45° is used. The device is equipped with two flat guide vanes. The rotor is operated at a speed of 1000 rev/min, and 220 g water is injected at a spraying rate of 20 g/min. After completion of injection, the rotor is operated at a reduced speed of 600 rev/min for a further 2 minutes.

In the subsequent drying stage the pellets are dried in a fluidized bed (Strea-1) at an inlet air temperature of 60° C. and an air flow level of 5 until the drying loss is less than 0.6%. Orange flavoring (0.01 g) is dissolved in demineralized water (10 ml); 5 ml is added during the drying stage.

### EXAMPLE 9

This example relates to the production of mannitol pellets containing povidone as binder and caffeine.

1143.75 g mannitol 60 and 93.75 g caffeine are mixed together in a shearing mixer (type Glatt VG-10) for 2 minutes. 12.50 g povidone (Collidon 30) is dissolved in 200 g demineralized water. The solution is added to the mixture in the shearing mixer within 5 minutes. The total running time of the shearing mixer is 7 minutes. A drying loss of about 11% is achieved. The shearing mixer is operated at the following settings; chopper (2000 rev/min); impeller (500 rev/min).

Then the mixture is transferred to a device as described previously. A rotor with a conical generated surface with a slope of 45° is used. The device is equipped with two flat guide vanes. The rotor is operated at a speed of 1000 rev/min, and 160 g water is injected at a spraying rate of 20 g/min. After completion of injection, the rotor is operated at a reduced speed of 600 rev/min for a further 2 minutes.

In the drying stage, the pellets are dried in a fluidized bed (GPCG) at an inlet air temperature of 70° C. and an inlet air volume of 50 m³/h until the drying loss is less than 0.6%.

### EXAMPLE 10

This example describes the production of a tablet by compressing quick-disintegrating mannitol pellets.

For pellet compression, a mixture is prepared with 1.2% magnesium stearate. For this, 98.8 g of mannitol pellets (500-800 μm) is mixed with 1.2 g magnesium stearate for 5 minutes in a Turbula mixer and then compacted to tablets of varying size in a Korsch EKO tablet press.

It is possible to make tablets from a mixture of the mannitol pellets and magnesium stearate. The tablets produced have the following dimensions:

| Diameter | Weight |
|---|---|
| (mm) | (mg) |
| 9 | 230 |
| 12 | 240 |

### EXAMPLE 11

The present example describes the production of pellets from mannitol and crospovidone, a swellable excipient. The pellets do not contain an active substance. For a person skilled in the art it is, however, immediately apparent that with corresponding application of the method described, for example to a composition that also contains an active substance as well as mannitol, pellets containing an active substance can be produced.

The mannitol is ground in an Alpine C 100 UPZ machine and is passed through a sieve with a mesh size of 0.8 mm. The crospovidone is passed through a sieve with a mesh size of 0.5 mm.

A mixture of 937.50 g (75 wt. %) mannitol and 312.50 g (25 wt. %) crospovidone is put in a shearing mixer (type Glatt VG-10). The apparatus is operated for 2 minutes, in order to break up aggregates. The settings are: chopper (2000 rev/min); impeller (100 rev/min). Then 550 g of saturated aqueous solution of mannitol is added. Addition takes place via a binary nozzle with a nozzle diameter of 1.2 mm at a spraying rate of 25 g/min and an atomizing air pressure of 1 bar. During this, the shearing mixer is operated at the settings stated above, to obtain a uniformly moistened material. This operation takes 24 min. The moistened mass obtained is passed through a sieve with a mesh size of 3.15 mm. The drying loss is 24.51%.

Then pellets are prepared from the moistened mass. For this, the mass is transferred to a device as described previously. A rotor with a conical generated surface with a slope of 30° is used. The device is equipped with two flat guide vanes. During operation, an air stream (75 m³/h) with an inlet air temperature of 30° C. and a dew point of 4.4° C. is passed through the bed of pellets that are forming. The rotor is operated at a speed of 800 rev/min. 445 g purified water is injected at a spraying rate of 22 g/min. After completion of injection, the rotor is operated at a reduced speed of 350 rev/min for a further 2 minutes. This operation takes 23 min. The drying loss is 31.19%.

The pellets thus obtained are dried in a fluidized-bed dryer (Glatt GPCG) at 60° C. air inlet temperature, until the product temperature exceeds 40° C. The drying loss is 1.81%.

The pellets obtained have a mean particle size of 0.35 mm (minimum: 0.17 mm; maximum: 0.43 mm).

### EXAMPLE 12

The present example describes the production of pellets from mannitol and crospovidone, a swellable excipient. The pellets do not contain an active substance.

For a person skilled in the art it is, however, immediately apparent that with corresponding application of the method described, for example to a composition that also contains an active substance as well as mannitol, pellets containing an active substance can be produced.

The mannitol is ground with an Alpine C 100 UPZ machine and is passed through a sieve with a mesh size of 0.8 mm. The crospovidone is passed through a sieve with a mesh size of 0.5 mm.

A mixture of 1125.00 g (90 wt. %) mannitol and 125.00 g (10 wt. %) crospovidone is put in a shearing mixer (type Glatt VG-10). The apparatus is operated for 2 minutes, in order to break up aggregates. The settings are: chopper (2000 rev/min); impeller (100 rev/min). Then 200 g of saturated aqueous solution of mannitol is added. Addition takes place via a binary nozzle with a nozzle diameter of 1.2 mm at a spraying rate of 47 g/min and an atomizing air pressure of 1 bar. During this, the shearing mixer is operated at the settings stated above, to obtain a uniformly moistened material. This operation takes 6 min. The moistened mass obtained is passed through a sieve with a mesh size of 3.15 mm. The drying loss is 11.21%.

Then pellets are prepared from the moistened mass. For this, the mass is transferred to a device as described previously. A rotor with a conical generated surface with a slope of 30° is used. The device is equipped with two flat guide vanes. During operation, an air stream (75 m³/h) with an inlet air temperature of 30° C. and a dew point of 6.2-6.7° C. is passed through the bed of pellets that are forming. The rotor is operated at a speed of 800 rev/min. 508 g purified water is injected at a spraying rate of 18 g/min. After completion of injection, the rotor is operated at a reduced speed of 350 rev/min for a further 2 minutes. This operation takes 31 min. The drying loss is 20.79%.

The pellets thus obtained are dried in a fluidized-bed dryer (Glatt GPCG) at 60° C. air inlet temperature, until the product temperature exceeds 42.5° C. The drying loss is 0.71%.

The pellets obtained have a mean particle size of 0.18 mm (minimum: 0.14 mm; maximum: 0.26 mm).

### EXAMPLE 13

The present example describes the production of pellets from mannitol, crospovidone, a swellable excipient, and citric acid.

The mannitol is ground in an Alpine C 100 UPZ machine and is passed through a sieve with a mesh size of 0.8 mm. The crospovidone is passed through a sieve with a mesh size of 0.5 mm.

A mixture of 1112.50 g (89 wt. %) mannitol, 125.00 g (10 wt. %) crospovidone and 12.50 g (1 wt. %) citric acid is put in a shearing mixer (type Glatt VG-10). The apparatus is operated for 2 minutes, in order to break up aggregates. The settings are: chopper (2000 rev/min); impeller (100 rev/min). Then 200 g of saturated aqueous solution of mannitol is added. Addition takes place via a binary nozzle with a nozzle diameter of 1.2 mm at a spraying rate of 45.5 g/min and an atomizing air pressure of 1 bar. During this, the shearing mixer is operated at the settings stated above, to obtain a uniformly moistened material. This operation takes 6 min. The moistened mass obtained is passed through a sieve with a mesh size of 3.15 mm. The drying loss is 10.70%.

Then pellets are prepared from the moistened mass. For this, the mass is transferred to a device as described previously. A rotor with a conical generated surface with a slope of 30° is used. The device is equipped with two flat guide vanes. During operation, an air stream (75 m³/h) with an inlet air temperature of 30° C. and a dew point of 4.8-7.4° C. is passed through the bed of pellets that are forming. The rotor is operated at a speed of 800 rev/min. 460 g purified water is injected at a spraying rate of 21 g/min. On completion of injection, the rotor is operated at a speed of 800 rev/min for a further 2 minutes. This operation takes 25 min. The drying loss is 21.09%.

The pellets thus obtained are dried in a fluidized-bed dryer (Glatt GPCG) at 60° C. air inlet temperature, until the product temperature exceeds 40.9° C. The drying loss is 0.89%.

The pellets obtained have a mean particle size of 0.43 mm (minimum: 0.26 mm; maximum: 0.52 mm). The particle size distribution was determined by sieve analysis with an ATM Sonic Sifter L3P (sieving time: 3 min, Amplitude of Sift and Pulse at setting 5). This is shown in FIG. 19.

### TEST EXAMPLES

Analytical methods are to be developed, with which the characteristics of the disintegration and melting behavior of pellets according to the invention can be determined. The disintegration characteristics are tested versus neutral pellets (Sugar Spheres). The following pellets are used in the test examples:

SDF E 0901, mannitol pellets

SDP E 0911, mannitol pellets with 1.5% PVP

SDF E 0919, mannitol pellets with 7.5% caffeine and 1.0% PVP

Nonpareils (batch 90183540X), neutral pellets

Nonpareils (batch 71762025X), neutral pellets

Particle sizes used: 250-500 μm, 500-800 μm and 800-1000 μm.

The pellets with the designations SDF E 0901, SDF E 0911 and SDF E 0919 were prepared in a corresponding manner, as described in the examples and reference examples given above.

### Test Example 1

The dissolution behavior of the pellets is tested in a small volume of liquid (0.5 ml demineralized water) and under mechanical loading, to represent the physiological conditions. The liquid volume corresponds to the volume of saliva at rest (without chewing or swallowing), and the mechanical loading corresponds to the movements of the tongue.

Varying amounts of pellets (200-800 mg) with various particle size distributions are put in 0.5 ml demineralized water in a Petri dish. The Petri dish is secured to the measuring cylinder holder of a tamped-volume measuring instrument. After 10 seconds, 30 tamping operations are carried out. Then for assessment, the shape or the appearance of the pellets is compared with the neutral pellets. FIGS. 1 to 12 show the disintegration behavior of quick-disintegrating pellets, and FIGS. 13 to 18 show conventional nonpareils. On comparing the figures, it can clearly be seen that quick-disintegrating pellets according to the present invention disintegrate into smaller fragments in the course of the test described, with visually discernible loss of pellet structure. In contrast, conventional nonpareils retain their shape. These pellets do not satisfy the disintegration criterion.

### Test Example 2

The disintegration time of the pellets in demineralized water (37° C.) is tested in the disintegration tester with perforated bottom. The amount of water is chosen such that the pellets are only immersed in water sufficiently for their whole surface to be wetted completely. The time to disintegration or dissolution of the pellets is measured.

Disintegration in a disintegration tester: Results

| | | | Disintegration |
|---|---|---|---|
| | Particle size | Quantity | time |
| Pellets | (μm) | (mg) | (min:s) |
| SDF E 0901 | 500-800 | 500 | 00:40-00:50 |
| SDF E 0911 | 500-800 | 500 | 00:40-01:00 |
| SDF E 0919 | 500-800 | 500 | 00:30-00:50 |
| Nonpareils | 600-710 | 500 | 01:30-02:00 |

The mannitol pellets tested disintegrate with loss of pellet structure within 10-20 seconds and then dissolve completely within a minute. The disintegration behavior of nonpareils is not comparable to that of the mannitol pellets investigated. They disintegrate/erode from their surface, becoming smaller. The neutral pellets retain their structure until complete disintegration. Moreover, the neutral pellets do not disintegrate until after about 2 minutes.

### Test Example 3

The melting behavior of the test pellets in the mouth is determined organoleptically by testers using an assessment scale.

To assess the disintegration of the pellets in the mouth, the sensation of a sample of 200 mg pellets of different particle sizes in the mouth after time intervals of 5, 10 and 20 seconds is determined with the aid of a scale. The mouth is rinsed with water between samples.

0=no sensation of a foreign body

1=paste-like, soft

2=sandy

3=gritty

The mannitol pellets investigated disintegrate into smaller, soft fragments, with loss of pellet structure. Neither the particle size nor the amount of all mannitol pellets tested has any significant role in the disintegration behavior. The pellets disintegrate independently of the amount used (200-800 mg) and particle size (250-1000 μm), without any substantial external differences being discernible.

The neutral pellets tested do not disintegrate in the organoleptic test, regardless of their size or amount. The pellets are also dimensionally stable at the end of the test.

The results confirm the melting behavior of the mannitol pellets and the accompanying sensation on the tongue. Whereas the mannitol pellets disintegrate quickly and feel soft on the tongue, nonpareils do not display comparable melting behavior. These feel hard and gritty on the tongue.

ORGANOLEPTIC TESTING OF MELTING BEHAVIOR BY 10 TESTERS
1. Assessment after 5 seconds

| | SDF E | | | | | | | | Non |
|---|---|---|---|---|---|---|---|---|---|
| | 0901 | | | | | | | | Pareils |
| | (250-500 | SDF E 0901 | SDF E 0901 | SDF E 0911 | SDF E 0911 | SDF E 0911 | Non Pareils | Non Pareils | 800-1000 |
| | μm) | 500-800 μm | 800-1000 μm | 250-500 μm | 500-800 μm | 800-1000 μm | 250-500 μm | 500-8001 μm | μm |
| Tester 1 | 1 | 1 | 2 | 1 | 1 | 2 | 3 | 3 | 3 |
| Tester 2 | 1 | 1 | 2 | 1 | 2 | 2 | 3 | 3 | 3 |
| Tester 3 | 1 | 1 | 2 | 1 | 1 | 1 | 3 | 3 | 3 |
| Tester 4 | 1 | 1 | 1 | 1 | 1 | 2 | 3 | 3 | 3 |
| Tester 5 | 1 | 1 | 1 | 1 | 1 | 1 | 3 | 3 | 3 |
| Tester 6 | 1 | 2 | 2 | 2 | 2 | 2 | 3 | 3 | 3 |
| Tester 7 | 1 | 1 | 1 | 1 | 1 | 1 | 3 | 3 | 3 |
| Tester 8 | 1 | 1 | 2 | 1 | 1 | 2 | 3 | 3 | 3 |
| Tester 9 | 1 | 1 | 2 | 1 | 1 | 2 | 3 | 3 | 3 |
| Tester 10 | 1 | 1 | 2 | 1 | 1 | 1 | 3 | 3 | 3 |
| Result | 10 × 1 | 9 × 1 | 3 × 1 | 9 × 1 | 8 × 1 | 4 × 1 | 10 × 3 | 10 × 3 | 10 × 3 |
| | | 1 × 2 | 7 × 2 | 1 × 2 | 2 × 2 | 6 × 2 | | | |
| Majority | 1 | 1 | 2 | 1 | 1 | 2 | 3 | 3 | 3 |
| Mean Value | 1 | 1.1 | 1.7 | 1.1 | 1.2 | 1.6 | 3 | 3 | 3 |
| | SDF E | | | | | | | | Non |
| | 0901 | | | | | | | | Pareils |
| | (250-500 | SDF E 0901 | SDF E 0901 | SDF E 0911 | SDF E 0911 | SDF E 0911 | Non Pareils | Non Pareils | 800-1000 |
| | μm) | 500-800 μm | 800-1000 μm | 250-500 μm | 500-800 μm | 800-1000 μm | 250-500 μm | 500-800 μm | μm |

2. Assessment after 10 seconds

| | | | | | | | | | |
|---|---|---|---|---|---|---|---|---|---|
| Tester 1 | 1 | 1 | 1 | 1 | 1 | 1 | 2 | 2 | 3 |
| Tester 2 | 0 | 1 | 1 | 1 | 1 | 2 | 3 | 3 | 3 |
| Tester 3 | 0 | 0 | 1 | 0 | 1 | 1 | 2 | 3 | 3 |
| Tester 4 | 1 | 1 | 1 | 0 | 0 | 1 | 2 | 3 | 3 |
| Tester 5 | 0 | 0 | 0 | 1 | 1 | 1 | 2 | 2 | 3 |
| Tester 6 | 0 | 1 | 1 | 1 | 1 | 1 | 3 | 3 | 3 |
| Tester 7 | 0 | 1 | 2 | 1 | 1 | 1 | 2 | 3 | 3 |
| Tester 8 | 1 | 1 | 1 | 1 | 1 | 2 | 2 | 2 | 3 |
| Tester 9 | 0 | 1 | 1 | 0 | 1 | 1 | 2 | 3 | 3 |
| Tester 10 | 0 | 0 | 1 | 1 | 1 | 1 | 3 | 3 | 3 |
| Result | 7 × O | 3 × O | 1 × O | 3 × O | 1 × O | 8 × 1 | 7 × 2 | 3 × 2 | 10 × 3 |
| | 3 × 1 | 7 × 1 | 8 × 1 | 7 × 1 | 9 × 1 | 2 × 2 | 3 × 3 | 7 × 3 | |
| | | | 1 × 2 | | | | | | |
| Majority | 0 | 1 | 1 | 1 | 1 | 1 | 2 | 3 | 3 |
| Mean value | 0.3 | 0.7 | 1 | 0.7 | 0.9 | 1.2 | 2.3 | 2.7 | 3 |

3. Assessment after 20 seconds

| | | | | | | | | | |
|---|---|---|---|---|---|---|---|---|---|
| Tester 1 | 0 | 0 | 0 | 0 | 0 | 0 | 2 | 2 | 3 |
| Tester 2 | 0 | 0 | 0 | 0 | 0 | 1 | 2 | 2 | 3 |
| Tester 3 | 0 | 1 | 0 | 0 | 1 | 1 | 1 | 2 | 3 |
| Tester 4 | 0 | 0 | 1 | 0 | 0 | 0 | 2 | 2 | 3 |
| Tester 5 | 0 | 0 | 1 | 0 | 0 | 0 | 1 | 2 | 3 |
| Tester 6 | 0 | 0 | 0 | 1 | 0 | 0 | 2 | 2 | 3 |
| Tester 7 | 0 | 0 | 0 | 0 | 0 | 0 | 2 | 2 | 2 |
| Tester 8 | 0 | 0 | 0 | 0 | 1 | 1 | 2 | 2 | 3 |
| Tester 9 | 0 | 0 | 0 | 0 | 0 | 0 | 2 | 2 | 3 |
| Tester 10 | 0 | 0 | 0 | 0 | 0 | 1 | 2 | 2 | 2 |
| Results | 10 × 0 | 9 × O | 8 × O | 9 × O | 8 × O | 6 × O | 2 × 1 | 10 × 2 | 2 × 2 |
| | | 1 × 1 | 2 × 1 | 1 × 1 | 2 × 1 | 4 × 1 | 8 × 2 | | 8 × 3 |
| Majority | 0 | 0 | 0 | 0 | 0 | 0 | 2 | 2 | 3 |
| Mean Value | 0 | 0.1 | 0.2 | 0.1 | 0.2 | 0.4 | 1.8 | 2 | 2.8 |

CRITERIA:
0 = NO SENSATION OF A FOREIGN BODY
1 = PASTE-LIKE, SOFT
2 = SANDY
3 = GRITTY

Assessment of melting behavior
Results (majority assessment by 10 testers):

| | Particle | Assessment | Assessment | Assessment |
|---|---|---|---|---|
| | size (μm) | (5 s) | (10 s) | (20 s) |
| SDF E 0901 | 250-500 | 1 | 0 | 0 |
| SDF E 0901 | 500-800 | 1 | 1 | 0 |
| SDF E 0901 | 800-1000 | 2 | 1 | 0 |
| SDF E 0911 | 250-500 | 1 | 1 | 0 |
| SDF E 0911 | 500-800 | 1 | 1 | 0 |
| SDF E 0911 | 800-1000 | 2 | 1 | 0 |
| Nonpareils | 250-500 | 3 | 2 | 2 |
| Nonpareils | 500-800 | 3 | 3 | 2 |
| Nonpareils | 800-1000 | 3 | 3 | 3 |

The mannitol pellets tested possess the property that after contact with a very small volume of liquid, within a short time they disintegrate into smaller particles, losing, their pellet structure. Neutral pellets from the prior art remain unchanged in the same test conditions. These different properties allow conclusions to be drawn concerning the actual disintegration behavior in the mouth.

In the disintegration tester, the mannitol pellets disintegrate within 20 seconds and dissolve within a minute. The neutral pellets disintegrate after about 2 minutes. The disintegration tester can indicate a time to complete disintegration. However, it is difficult to draw conclusions about the actual melting behavior and the resultant sensation on the tongue, because in this test the amount of liquid is far greater and therefore does not reflect the physiological conditions in the mouth.

By means of organoleptic testing it is possible to characterize the difference in behavior of the mannitol pellets versus nonpareils. The mannitol pellets disintegrate after a few seconds to a soft mass, which is not perceived as unpleasant or even disturbing. In comparison, nonpareils are perceived as gritty particles, which are even still perceived as unpleasant, when the mannitol pellets tested have already disintegrated completely and are no longer perceptible.

1. Pellet with a length/width ratio of less than about 1.4, which comprises a core that contains a water-soluble excipient and optionally an active substance homogeneously distributed therein.

2. Pellet according to claim 1, characterized in that the excipient is selected from water-soluble carbohydrates.

3. Pellet according to claim 1, characterized in that the excipient comprises mannitol.

4. Pellet according to claim 1, which additionally contains a binder.

5. Pellet according to claim 1, which additionally contains a water-swellable excipient.

6. Pellet according to claim 1, additionally containing one or more constituents selected from the group comprising taste improving agents, aromatics, colorants, preservatives, physical stabilizers, chemical stabilizers, antiadherents, disintegration accelerators and disintegration inhibitors.

7. Pellet according to claim 1, which disintegrates into smaller fragments with visually discernible loss of pellet structure, when the following test is carried out: (a) 200 to 800 mg pellets are put in a Petri dish with 0.5 ml demineralized water, (b) immediately thereafter, the Petri dish is secured to the measuring cylinder holder of a tamped-volume measuring instrument, (c) after 10 seconds, 30 tamping operations are carried out, (d) then the pellets are photographed immediately.

8. Pelletized product, comprising a plurality of pellets, of which at least 90% comply with the definition according to claim 1.

9. Pelletized product according to claim 7, characterized in that the disintegration time measured in a disintegration tester with a perforated bottom is less than 1:30 min.

10. Pelletized product according to claim 7, which in an organoleptic test with the classifications
0=no sensation of a foreign body
1=paste-like, soft
2=sandy
3=gritty
is classified by the majority of at least 10 testers at most as 2=sandy when assessed after 5 seconds.

11. Pelletized product according to claim 9, which is classified by the majority of at least 10 testers at most as 1=paste-like, soft, when assessed after 10 seconds.

12. Pelletized product according to claim 9, for which the majority of at least 10 testers no longer perceive a sensation of a foreign body (classification=0), when assessed after 20 seconds.

13. Pelletized product according to claim 7, further comprising one or more constituents selected from the group comprising agents for improving taste and aromatics.

14. Pelletized product according to claim 7 for sublingual administration.

15. Use of pellets according to claim 1 for the production of a pharmaceutical preparation.

16. Tablet, obtainable by compaction of pellets according to claim 1.

17. Method of production of a pellet according to claim 1, which comprises the following stages:
(a) preparation of a powdered starting material;
(b) pelletization of the powdered starting material using a pharmaceutically acceptable liquid diluent;
(c) drying of the pellet obtained.

18. Use of a pelletized product according to claim 7 for the production of a pharmaceutical preparation.

19. Method of production of a pelletized product according to claim 7, which comprises the following stages:
(a) preparation of a powdered starting material;
(b) pelletization of the powdered starting material using a pharmaceutically acceptable liquid diluent;
(c) drying of the pellet obtained.
